(19) United States
(12) Patent Application Publication
(10) Pub. No.: US 2017/0273832 A1
(43) Pub. Date: Sep. 28, 2017

(54) Compression Stocking with an Adjustable Pressure Arrangement

(71) Applicant: Moutaa BenMaamer, Fort Myers, FL (US)

(72) Inventor: Moutaa BenMaamer, Fort Myers, FL (US)

(21) Appl. No.: 15/387,255

(22) Filed: Dec. 21, 2016

Related U.S. Application Data
(60) Provisional application No. 62/313,369, filed on Mar. 25, 2016.

(51) Int. Cl. A61F 13/08

(52) U.S. Cl. CPC A61F 13/085; A61F 2013/0028

(57) ABSTRACT

A compression stocking with the ability to change the applied pressure contains a foot portion, a calf portion, and a slit. The slit functions as a size-adjustable opening so that the compression stocking can be worn and removed conveniently. The size of the slit is controlled by at least one cord which is tethered around a first set of pulleys and a second set of pulleys. When the pressure applied by the foot portion and the calf portion needs to varied, a user adjusts the cord around the first set of pulleys and the second set of pulleys so that the overall pressure is changed. By utilizing a pressure sensing device, the exact pressure can be measured. Moreover, by using a cord retention device the probability of the cord loosening around the leg can be eliminated.

The current application claims a priority to the U.S. Provisional patent application Ser. No. 62/313,369 filed on Mar. 25, 2016.

### FIELD OF THE INVENTION

The present invention relates generally to the field of compression socks. More specifically, the present invention relates to a compression sock that addresses a venous return problem.

### BACKGROUND OF THE INVENTION

Venous return is the rate of blood flow back to the heart. When medical conditions related to venous return occurs, compression therapy is generally used to solve the issue. In doing so, compression therapy is applied externally to the lower leg to increase pressure on the skin to counteract the force of gravity. Bandage components wrapped around the leg and compression hosiery are generally used in the process of applying compression to the lower leg. Even though existing compression hosiery addresses the issue at hand to a certain extent, there are certain disadvantages too.

One of the main disadvantages with existing compression stockings is the fixed size. The fixed size requires the user to stretch the body of the compression stocking in the process of inserting the leg. This at times can be extremely stressful due to the material properties of compression stocking. As an example, a user who is suffering from a medical condition such as arthritis can have great difficulty stretching a compression stocking appropriately. The difficulty in wearing can be stressful when attempting to wear the compression stocking within a short time period.

The fixed size leads to another major drawback of compression stockings, which is fixed pressure. As an example, the pressure applied at the leg can be either too much or too little. Therefore, an effective method that can adjust the pressure accordingly is clearly needed. Compression stockings with zippers addresses the issue to some extent but does not fully resolve the issue.

The objective of the present invention is to address the aforementioned issues. More specifically, the present invention addresses the issues related to wearing compression socks and issues related to fixed pressure. In doing so, the present invention introduces an effective design wherein the user can conveniently insert the leg into the compression stocking and also adjust the pressure of the stocking as preferred. Therefore, by utilizing the present invention a user experiencing venous return problems can obtain the expected results with minimum effort.

### BRIEF DESCRIPTION OF THE DRAWINGS

FIG. 1 is a perspective view of the present invention being worn on a leg.

FIG. 2 is a side view of the present invention being worn on the leg.

FIG. 3 is a front view of the present invention being worn on the leg.

FIG. 4 is a rear view of the present invention being worn on the leg.

FIG. 5 is a front view of the present invention, wherein the cord retention device is also illustrated.

FIG. 6 is a front view of the present invention, wherein the pressure sensing device is also illustrated.

### DETAIL DESCRIPTIONS OF THE INVENTION

All illustrations of the drawings are for the purpose of describing selected versions of the present invention and are not intended to limit the scope of the present invention.

The present invention introduces a compression stocking that can be easily worn and has an adjustable pressure arrangement. More specifically, the present invention has a lace arrangement that is used with a set of pullies so that the size of the compression stocking and the pressure applied by the compression stocking can be adjusted.

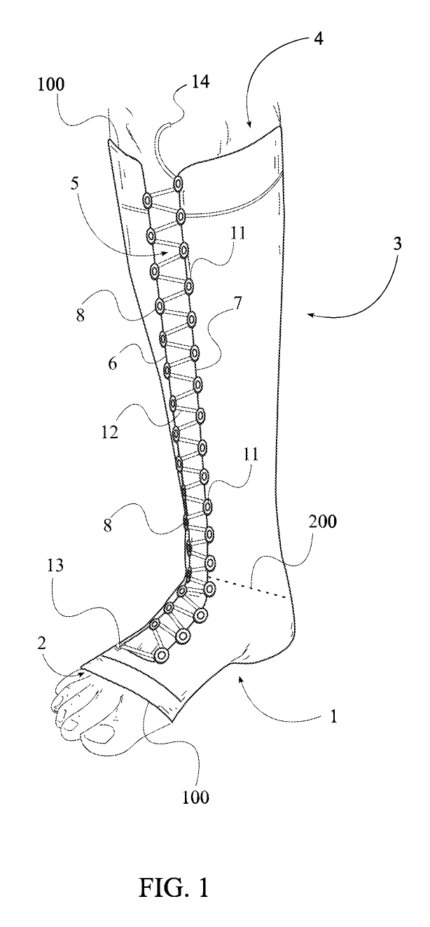

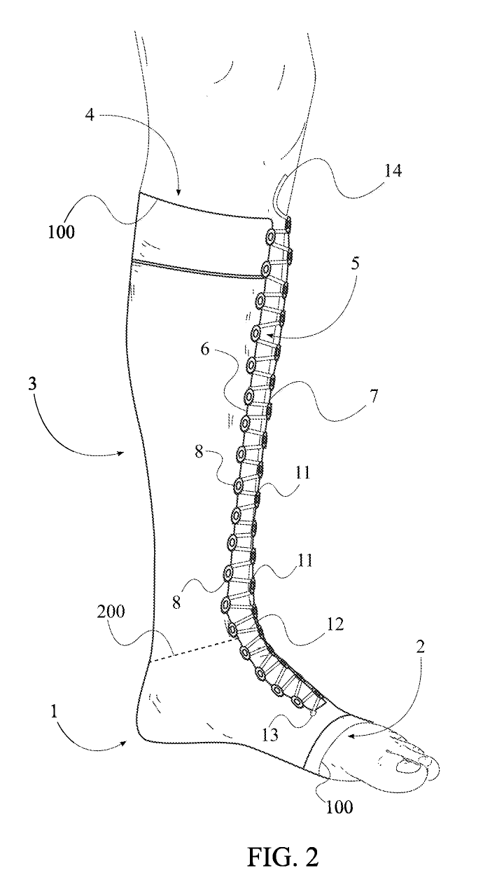

Compression stockings are generally worn so that the calf muscle and the foot of a user is covered. In order to serve the standard requirements of a traditional compression stocking, the present invention comprises a foot portion 1 and a calf portion 3 as seen in FIG. 1. The calf portion 3 comprises a calf opening 4 so that the user can conveniently wear the present invention. As seen in FIG. 2, the foot portion 1 covers an area around the ankle of the user. The foot portion 1 is connected adjacent to the calf portion 3 and opposite the calf opening 4 so that the foot and the calf can be simultaneously compressed with the use of the present invention. More specifically, the calf portion 3 is connected to the foot portion 1 creating an L-shaped body. The present invention further comprises a slit 5, a first set of pulleys 8, a second set of pulleys 11, and an at least one cord 12. The slit 5, which is a size adjustable opening, allows the user to adjust the size of the compression stocking prior to wearing and after being worn by the user. In order to do so, the slit 5 traverses from the calf opening 4 along the calf portion 3 and into the foot portion 1. The positioning of the slit 5 allows the user to compress both the calf portion 3 and the foot portion 1 simultaneously. The at least one cord 12 along with the first set of pulleys 8 and the second set of pulleys 11 are used to adjust the size of the slit 5 and resultantly adjust the pressure applied by the compression stocking. In addition to adjusting the pressure applied by the compression stocking, the at least one cord 12 is also beneficial in the process of wearing and removing the compression stocking from the leg. As an example, the at least one cord 12 is controlled so that the user can insert the leg through the calf opening 4. When inserted, the user can adjust the at least one cord 12 so that the required pressure is applied on the leg of the user.

For the slit 5 to be used for size adjusting purposes, the slit 5 comprises a first edge 6 and a second edge 7. The first edge 6 and the second edge 7 are positioned opposite to each other across the slit 5. As mentioned before, the overall size of the present invention is adjusted by manipulating the size of the slit 5. When considering the positioning, the first set of pulleys 8 is equidistantly distributed along the first edge 6. Similarly, the second set of pulleys 11 is equidistantly positioned along the second edge 7. To allow the user to adjust the size of the slit 5 with the use of the at least one cord 12, the first set of pulleys 8 and the second set of pulleys 11 are tensionably tethered with the at least one cord 12. Therefore, by controlling the at least one cord 12, the slit 5 can be adjusted and therefore the overall size of the present invention and the pressure applied by the present invention can also be adjusted. In the preferred embodiment of the present invention, the first set of pulleys 8 and the second set of pulleys 11 are alternatingly positioned with each other along the slit 5. However, the first set of pulleys 8 and the second set of pulleys 11 can be positioned differently in other embodiments of the present invention. As an example, the second set of pulleys 11 can be positioned directly opposite the first set of pulleys 8.

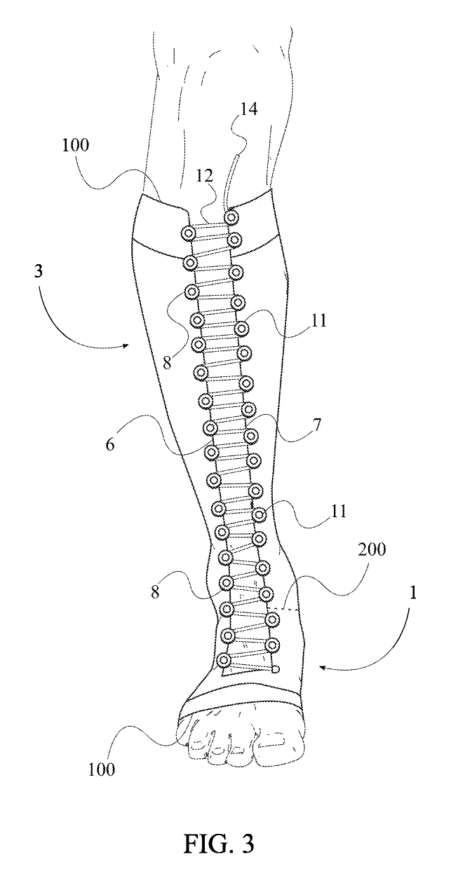

In different embodiments of the present invention, the at least one cord 12 can be configured around the first set of pulleys 8 and the second set of pulleys 11 differently. As seen in FIG. 3, in the preferred embodiment of the present invention, the at least one cord 12 is alternatingly tethered between the first set of pulleys 8 and the second set of pulleys 11 along the slit 5. However, in different embodiments of the present invention, the at least one cord 12 can be tethered differently.

The foot portion 1 and the calf portion 3 each comprise a first rim 100 and a second rim 200. More specifically, a body of the foot portion 1 extends from the first rim 100 of the foot portion 1 to the second rim 200 of the foot portion 1. On the other hand, a body of the calf portion 3 extends from the first rim 100 of the calf portion 3 to the second rim 200 of the calf portion 3. Since the foot portion 1 needs to be connected adjacent to the calf portion 3, the second rim 200 of the foot portion is connected to the second rim 200 of the calf portion 3. Alternatively, the foot portion 1 and the calf portion 3 share the same second rim 200. When comparing the positioning of the slit 5 with respect to the first rim 100 and the second rim 200, the slit 5 traverses through the calf portion 3 from the first rim 100 of the calf portion 3 to the second rim 200 of the calf portion 3. Moreover, the slit 5 traverses into the foot portion 1 at the second rim 200 of the foot portion 1. In other words, the slit 5 extends from the first rim 100 of the calf portion and into the foot portion 1.

The present invention can be available as a closed-toe compression stocking or an open-toed compression stocking. To provide user comfort and convenience when utilizing the present invention, the foot portion 1 comprises a foot opening 2. The foot opening 2 allows the toe of the user to be exposed when the present invention is worn. As illustrated, the foot opening 2 is positioned opposite the calf portion 3 and along the foot portion 1 such that the foot opening 2 is delineated by the first rim 100 of the foot portion 1. Similar to delineating the foot opening 2, the first rim 100 of the calf portion 3 delineates the calf opening 4. When considering the positioning of the slit 5, the slit 5 is positioned adjacent to the foot opening 2 but separated from the foot opening 2 by a portion of the body of the foot portion 1. The size of both the foot opening 2 and the calf opening 4 can be controlled by adjusting the tension of the at least one cord 12.

The at least one cord 12 comprises a first end 13 and a second end 14. The first end 13 is mounted onto the foot portion 1 and acts as an anchor point to the at least one cord 12. The second end 14 is positioned adjacent to the calf opening 4. Since the first end 13 is mounted onto the foot portion 1, the second end 14 is used when the user needs to adjust the pressure applied by the present invention. The first end 13 is positioned adjacent a first pulley 9 of the first set of pulleys 8. When appropriately positioned, the first end 13 is mounted onto the foot portion 1 adjacent to the first rim 100 of the foot portion 1. On the other hand, the second end 14 is positioned adjacent a last pulley 10 of the first set of pulleys 8. When positioned, the second end 14 is positioned adjacent to the second rim 200 of the calf portion 3. The positioning of the first end 13 and the second end 14 ensures that the slit 5 can be effectively controlled with the use of the at least one cord 12. As an example, if the user intends on increasing the pressure applied by the present invention, the user holds and pulls the second end 14 until the preferred pressure is applied on the leg.

In different embodiments of the present invention, the first end 13 can be mounted onto the foot portion 1 differently. In one embodiment of the present invention, the first end 13 can be fixed to the foot portion 1. Therefore, the at least one cord 12 will always be attached to the foot portion 1 by the first end 13. In another embodiment of the present invention, the first end 13 can be removably attached to the foot portion 1. This allows the user to completely detach the at least one cord 12 from the foot portion 1. The detachability can be advantageous if the user intends on interchanging the at least one cord 12 over time.

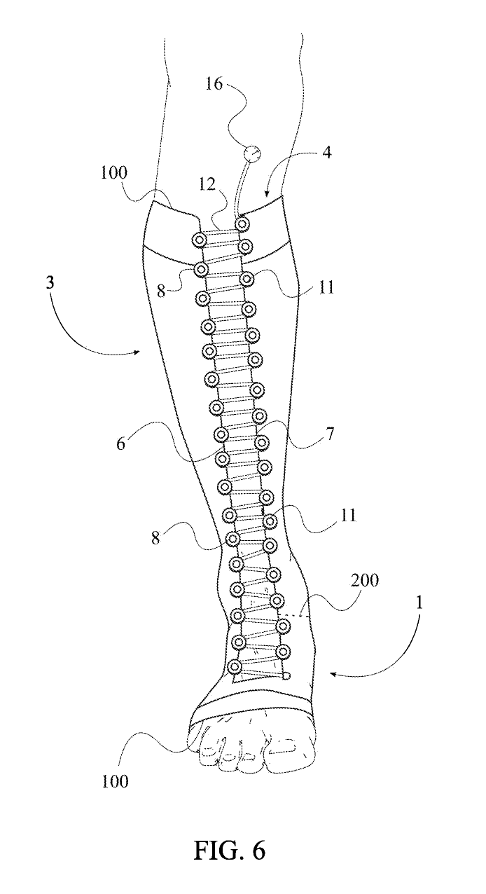

In a different embodiment, the present invention can comprise a pressure sensing device 16. As illustrated in FIG. 6, the pressure sensing device 16 is engaged to the second end 14 so that the pressure applied on the leg by pulling on the second end 14 is displayed through the pressure sensing device 16. To do so, the pressure sensing device 16 is externally positioned adjacent the calf opening 4. As an example, if the user needs to apply a medically recommended pressure on the leg, the pressure sensing device 16 can be used to adjust the pressure to the recommended pressure.

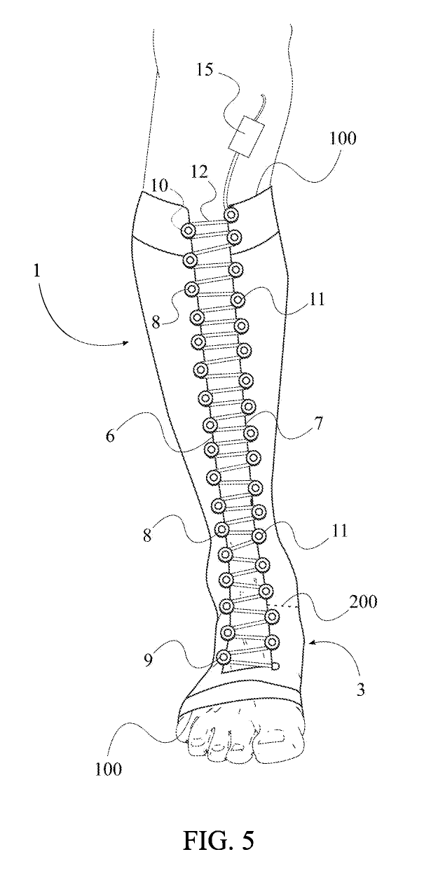

As illustrated in FIG. 5, in one embodiment, the present invention can comprise a cord retention device 15 that prevents the at least one cord 12 from loosening around the leg of the user. Similar to the pressure sensing device 16, the cord retention device 15 will also be positioned adjacent the calf opening 4 and the second end 14 is positioned into the cord retention device 16. Therefore, the cord retention device 16 can be effectively used to hold the at least one cord 12 stationary when worn by the user.

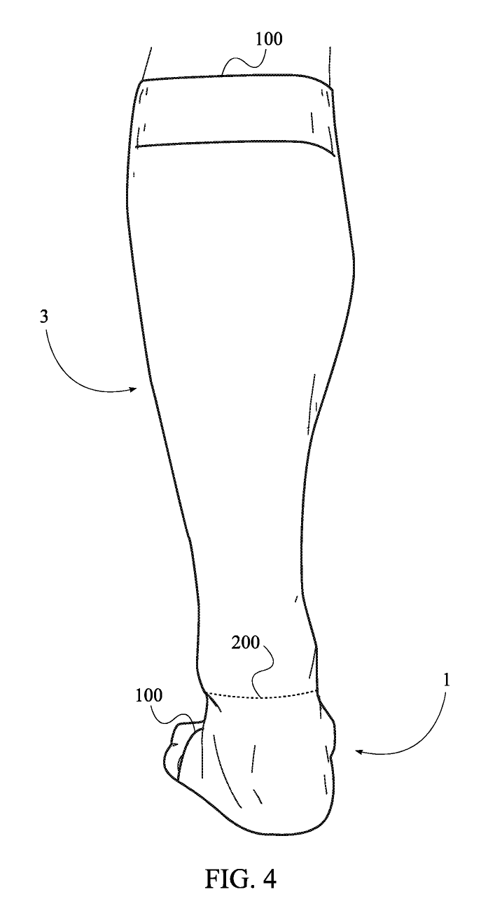

When utilizing the present invention, the following process flow is generally followed. Initially, the size of the slit 5 is adjusted by controlling the at least one cord 12. By adjusting the size of the slit 5, the user also adjusts the calf opening 4 and the foot opening 2. If a second cord is also available, both the at least one cord 12 and the second cord are controlled to adjust the size of the slit 5. More specifically, the slit 5 is adjusted so that the user can comfortably wear the present invention. As seen in FIG. 4, the calf portion 3 is appropriately positioned around the calf muscle of the user and the foot portion 1 is appropriately positioned around the foot of the user. Next, the user pulls the second end 14 so that the calf portion 3 and the foot portion 1 are adjusted to apply pressure onto the leg of the user. More specifically, the at least one cord 12 is adjusted so that the slit 5 is adjusted, which in turn adjusts the pressure applied on the leg. If the second cord is also available, the second cord is also adjusted similar to adjusting the at least one cord 12. The pressure applied on the leg is measured with the pressure sensing device 17. Next, when the preferred pressure is applied, the second end 14 is secured in place with the cord retention device 16. When the user needs to remove the present invention, the at least one cord 12 along with the first set of pulleys 8 and the second set of pulleys 11 are used to expand the size of the slit 5. When the slit 5 is appropriately expanded, the calf opening 4 and the foot opening 2 are also expanded so that the present invention can be conveniently removed from the leg.

Although the invention has been explained in relation to its preferred embodiment, it is to be understood that many other possible modifications and variations can be made without departing from the spirit and scope of the invention as hereinafter claimed.

What is claimed is:

1. A compression stocking with an adjustable pressure arrangement comprises:
a foot portion;
a calf portion;
a slit;
a first set of pulleys;
a second set of pulleys;
at least one cord;
the calf portion comprises a calf opening;
the slit being delineated by a first edge and a second edge;
the foot portion being connected adjacent to the calf portion opposite the calf opening;
the slit traversing from the calf opening along the calf portion into the foot portion;
the first set of pulleys being distributed equidistantly along the first edge;
the second set of pulleys being distributed equidistantly along the second edge; and
the first set of pulleys and the second set of pulleys being tensionably tethered with the at least one cord.

2. The compression stocking with an adjustable pressure arrangement as claimed in claim 1, wherein the first set of pulleys and the second set of pulleys are alternatingly positioned with each other along the slit.

3. The compression stocking with an adjustable pressure arrangement as claimed in claim 1, wherein the at least one cord is alternatingly tethered between the first set of pulleys and the second set of pulleys along the slit.

4. The compression stocking with an adjustable pressure arrangement as claimed in claim 1 comprises:
the foot portion and the calf portion each comprise a first rim and a second rim;
the slit traversing through the calf portion from the first rim to the second rim;
the slit traversing into the foot portion at the second rim; and
the second rim of the foot portion being connected to the second rim of the calf portion.

5. The compression stocking with an adjustable pressure arrangement as claimed in claim 4 comprises:
the foot portion comprises a foot opening;
the foot opening being positioned opposite the calf portion along the foot portion;
the slit being positioned adjacent the foot opening;
the foot opening being delineated by the first rim of the foot portion; and
the calf opening being delineated by the first rim of the calf portion.

6. The compression stocking with an adjustable pressure arrangement as claimed in claim 1 comprises:
the at least one cord comprises a first end and a second end;
the first end being mounted onto the foot portion adjacent to the first rim of the foot portion; and
the second end being positioned adjacent to the second rim of the calf portion.

7. The compression stocking with an adjustable pressure arrangement as claimed in claim 6, wherein the first end is fixed to the foot portion.

8. The compression stocking with an adjustable pressure arrangement as claimed in claim 6, wherein the first end is removably attached to the foot portion.

9. The compression stocking with an adjustable pressure arrangement as claimed in claim 1 comprises:
a cord retention device;
the at least one cord comprises a first end and a second end;
the cord retention device being positioned adjacent the calf opening; and
the second end being positioned into the cord retention device.

10. The compression stocking with an adjustable pressure arrangement as claimed in claim 1 comprises:
a pressure sensing device;
the at least one cord comprises a first end and a second end;
the pressure sensing device being externally positioned adjacent the calf opening; and
the pressure sensing device being engaged with the second end.

11. The compression stocking with an adjustable pressure arrangement as claimed in claim 1, wherein the calf portion is connected to the foot portion in an L-shape.
